(19) United States
(12) Patent Application Publication
(10) Pub. No.: US 2019/0133804 A1
(43) Pub. Date: May 9, 2019

(54) ANKLE SUPPORT

(71) Applicant: Mariti Antonio MUNOZ, Brownsville, TX (US)

(72) Inventor: Mariti Antonio MUNOZ, Brownsville, TX (US)

(21) Appl. No.: 16/151,800

(22) Filed: Oct. 4, 2018

Related U.S. Application Data
(60) Provisional application No. 62/583,215, filed on Nov. 8, 2017.

(51) Int. Cl. A61F 5/01

(52) U.S. Cl. CPC A61F 5/0111

(57) ABSTRACT

An ankle support article has a body with a generally cylindrical configuration and adapted to conform to a shape of the human ankle. The body has a first portion and a second portion integrally formed together of a flexible material. The first portion is adapted to extend over a heel portion of the human foot. The second portion is adapted to extend over the human ankle and over a portion of a human calf. The body has a slot formed between edges at a forward part of the body. An endoskeletal structure is affixed to the body and extends along sides of the first and second portions of the body and across a bottom of the first portion of the body.

### CROSS-REFERENCE TO RELATED APPLICATIONS

The present application claims priority from U.S. Provisional Application Ser. No. 62/583,215, filed on Nov. 8, 2017, and entitled “Ankle Support”.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

### 1. Field of the Invention

The present invention relates to ankle supports. More particularly, the present invention the relates to ankle supports for supporting and protecting the ankle and associated parts of the human foot. More particularly, the present invention the relates to ankle supports that enhance the flexibility of the foot and ankle while, at the same time, providing comfort and support to other areas of the ankle and foot.

### 2. Description of Related Art Including Information Disclosed Under 37 CFR 1.97 and 37 CFR 1.98

The complex interactions between bones, ligaments, tendons and their musculature enable the foot to transfer from a mobile adaptor during ground interface to a rigid lever for propulsion. The body requires a flexible foot to accommodate the variations in the external environment, a semi-rigid foot that can act as a spring or lever arm for push off during gait and a rigid foot to enable body weight to be carried with adequate stability. The position of the subtalar joint is the major factor in this adaptation. Eversion of the calcaneus results in pronation of the subtalar joint which in turn “unlocks” the transverse tarsal joint-whilst inversion leads to supination of the subtalar joint which in turn “locks” the transverse tarsal joint creating a rigid forefoot. A thorough understanding of the interrelation between joint axes and planes of motion is essential to analysis of compensatory motion as a consequence of pathological or restricted motion.

The bones of the foot interlock to form three distinct arches. The medial and lateral longitudinal arches and the transverse arch. The formation of these arches allows the support of body weight with the least expenditure of anatomical material and provides protection of the nerves and vasculature of the plantar foot. The bones of the medial arch are passively interlocked by the plantar aponeurosis, the long and short plantar ligaments, and the plantar calcaneonavicular (spring) ligament. During weight bearing, and heel raise, tension is increased especially in the plantar aponeurosis as the metatarsophalangeal joint extends adding stability.

The shape of the articular surfaces is particularly important to the evaluation of components of joint motions. Ligaments guide and check excessive joint motion with fiber direction determining what motions are guided and limited. The dorsal surface of the talus is called the trochlea. It is convex anterior-posterior, and convex-concave-convex medial to lateral. The trochlea has been described as a section of a cone with its base on the medial side. This surface articulates with the reciprocally shaped distal tibia. The lateral aspect of the talus is flat and is directed slightly medially from anterior to posterior and articulates with a reciprocally shaped distal fibula. The talus is slightly wider anteriorly than posteriorly.

The distal tibiofibula joint forms a deepened joint called the ankle mortise. The ligaments which stabilize the ankle consist of the strong medial ligament, and the three bands of the lateral ligament, together with the lateral malleolus provide lateral stability to the ankle joint and stabilize the talus within the ankle mortise.

Rotary stability in a horizontal plane is provided by tension in the collateral ligaments, by compression of the talar facets against the malleoli and, in the loaded state, by the shape of the articular surfaces. The horizontal components of tension in the collateral ligaments form a ring resisting rotation by tension in opposing pairs. The primary function of the malleoli as far as stability is concerned, is to serve as pillars so that the collateral ligaments are close to the ankle axis and hence permit plantar-dorsiflexion. Rotary instability may be an additional factor in patients whose symptoms persist after injury to the lateral ligaments.

The transverse tarsal joint exhibits triplanar motion and consists of the talonavicular and calcaneocuboid joints. Motion perpendicular to the two axes may be independent of each other, but both are dependent upon the position of the subtalar joint.

The calcaneocuboid axis lies close to the longitudinal axis and is directed anteriorly and superiorly 15 degrees to the horizontal and 9 degrees to the longitudinal, whilst the calcaneonavicular joint is oblique, directed anteriorly and superiorly at 52 degrees to the horizontal and medially at 57 degrees to the longitudinal axis.

During activity, as a person lands from a jump, the ankle is plantar flexed. The three lateral ligaments and peroneal muscle provide the primary restraints to ankle inversion. As inversion proceeds and the strength of the peroneal muscle is overcome, injury to the lateral ligaments will occur as the range of motion of the talus in the ankle mortis is exceeded.

Injury to the lateral ligaments occurs in a progressive fashion. The anterior talofibular ligament being the first to be injured. If inversion continues the calcaneofibular ligament is the next to be injured and finally, in severe cases, the posterior talofibular ligament is torn. The other mechanism of ankle injury is eversion. Eversion injury is more severe than inversion injury and may lead to chronic ankle instability.

Previous devices such as taping strapping and semi-rigid orthosis have been proposed for supporting these joints by rigidly (or semi-rigidly) inhibiting motion in the direction of possible injury. The ankle is prone to the following types of injury: inversion, eversion, dorsi flexion sprains and plantar flexion sprains.

Strapping of the ankle has been used to support the ankle. This involves taping of the ankle with non-elastic adhesive tape. Cloth wraps, elastic wraps and elastic adhesive materials have also been used. Whether taping, regardless of material used, is effective is debatable. Tape initially provides restriction of motion but this diminishes as exercise progresses. Taping is effective in preventing ankle sprains. Tape is not reusable and for taping to be effective the ankle may need to be retaped which involves unwinding and re-winding of the tape which is time consuming and expensive.

Ankle supports or braces which are re-usable have attempted to support the ankle by rigidly or semi-rigidly inhibiting motion in the direction of possible injury.

In the past, various patents of issued with respect to ankle supports. An early patent was U.S. Pat. No. 2,830,585, issued on Apr. 15, 1958 to E. I. Weiss. This patent describes an ankle support having a body portion adapted to encircle the ankle at the ankle bone. The body includes a stirrup member that passes under the foot of the wearer and a detachable fastener for adjustably securing the front portions of the support to each other. The body portion is provided with a pair of pneumatic cushions adapted to rest against each ankle bone on either side of the Achilles tendon of the wearer.

U.S. Pat. No. 3,506,000, issued on Apr. 14, 1970 to J. R. Baker, describes an ankle support having a body member that extends around the Achilles tendon at the back of the foot along the inside and outside of the foot with a band at its upper edge to support it in position and with stretchable straps supported in positions to the body member and extending in opposite directions. The straps have a length suitable for passing under the arch of the foot and over the instep of the foot in opposite directions to one another and then about the leg in opposite directions so as to overlap. A means is provided for holding these portions in an overlapping relationship.

U.S. Pat. No. 3,584,622, issued on Jun. 15, 1971 to A. J. Domenico, provides a support device for preventing ankle injuries. The support device has a support member of a flexible sheet material including a foot-receiving member adapted to receive a rear portion of a wearer's foot and to cover the ankle. A stirrup portion extends upwardly from the foot-receiving member and is adapted to contact opposite sides of the wearer's leg above the ankle. No part of the foot-receiving member intrudes into the area of the calf muscles of the wearer so that restriction of movement of the Achilles tendon is avoided. No part of the foot-receiving member extends forwardly beyond the metatarsus of the wearer's foot. The forward and upper portions of the foot-receiving member open to avoid restriction of desired movement.

U.S. Pat. No. 3,674,023, issued on Jul. 4, 1972 to R. C. Mann discloses an ankle support that has an open-fronted heel boot of relatively stiff construction so as to brace an ankle against sprains without constricting the ankle. An elastic strap section spans the wearer's instep. A non-elastic ankle-wrapping strap section holds the boot snugly on the wearer's foot without significantly restricting walking movements.

U.S. Pat. No. 4,597,395, issued on Jul. 1, 1986 to Barlow et al., teaches and ankle support that assists in providing comfort and support to the tissues of the human ankle. The support includes inter-connected dorsal, plantar and heel straps forming a heel block that can be fitted to the heel area of the foot. A crossover strap is connected to the heel strap and includes free ends that can be extended from behind the heel and be releasably secured to the heel block forward of the ankle. Fastener members are provided on the heel block and on ends of the crossover strap to secure the crossover strap in its operative position. The fastener members are attached to end of the straps.

U.S. Pat. No. 4,865,023, issued on Sep. 12, 1989 to Craythorne et al., shows an ankle support apparatus which may be worn separately or incorporated into footwear. The ankle support device has a bracing pad which seats on the lateral side of the foot on one portion of the heel bone and braces against the underside of the lateral malleolus. The bracing pad is secured to the foot by a stirrup strap which attaches at the lower end to an ankle cuff and at the lower end to a heel cup. The pad is anatomically configured to support the lateral side of the heel bone and the inferior side of the lateral malleolus.

U.S. Pat. No. 5,496,263, issued on Mar. 5, 1996 to Fuller et al., provides an ankle stabilization system. This ankle stabilization member includes an ankle brace with an upper calf member hingedly connected to a lower foot member and a plurality of reinforced areas for strengthening and supporting a region around the talus bone. The reinforced areas are located above and below the hinge or in the approximate vicinity of the ankle and talus bone. The hinge is located on outwardly bowed cup-shaped portion of the ankle brace for comfortably receiving the mallseli of the user. The upper member includes a pair of arcuate elements slidable with respect to one another to adjust the opening formed thereby for various calf sizes. The lower member is designed to reduce stiffness in areas such as the front of the foot. Several straps are provided to securely couple the upper member and the lower member to the user.

U.S. Pat. No. 5,833,640, issued on Nov. 10, 1998 to Vazquez et al., teaches an ankle and foot support brace which is made of an inner slide-on sleeve, a plantar cushion, and an outer mono-unit strapping system. The sleeve is made of a stretchable resilient material. The plantar cushion may be removably or permanently attached to the brace so as to form a therapeutic orthotic.

U.S. Pat. No. 5,944,678, issued on Aug. 31, 1999 to C. J. Hubbard, provides an ankle brace having an upper portion adapted to extend around a wearer's ankle above the malleoli. The upper portion has at least one anchor site. A foot section conforms to a portion of the sole of the wearer's foot and an elastic lateral connection extends between the foot section and the upper portion so as to enable a normal range of movement of the ankle. The anchor site has a pad with holes to which the skin of the foot conform so as to adhere the pad to the skin.

U.S. Pat. No. 6,083,184, issued on Jul. 4, 2000 to M. J. Kenosh, teaches an ankle orthotic of substantially resilient material. This ankle orthotic has a heel portion, an ankle support portion, and a talofibular support portion. The heel portion is substantially rigid and form fitting so as to prevent excessive inversion forces from being generated at the subtalar joint. The ankle support portion is relatively resilient and extends vertically from the heel portion, with the medial aspect extending higher than the lateral aspect. The proximal end of the ankle support portion is enclosed via a fastening strap. The talofibular support portion extends laterally and distally from the heel portion and reinforces the relatively vulnerable anterior talofibular ligament.

U.S. Pat. No. 6,602,215, issued on Aug. 5, 2003 to D. H. Richie, shows an ankle brace having a lateral upright limb member and a medial upright limb member to which a footplate/stirrup is pivotally secured. The brace includes a medial sling mechanism having a medial lifting strap secured to the stirrup at the location of the medial arch and arranged to be extended over the wearer's foot. A lateral sling mechanism is also provided. The lateral sling mechanism includes a lateral lifting strap secured to the stirrup at the location of the lateral arch and slightly posteriorly of the medial sling strap. The lateral lifting strap is arranged to be extended over the wearer's foot to releasably connect to the medial upright. The footplate/stirrup includes a pair of recesses into which portions of the respective lifting straps extend.

It is an object of the present invention to provide an ankle support that effectively supports the ankle.

It is another object of the present invention to provide an ankle support that provides optimal flexibility to the ankle and foot.

It is another object of the present invention to provide an ankle support that can be easily applied to the foot and ankle.

It is another object of the present invention to provide an ankle support that conforms to the shape of the ankle and foot.

It is a further object of the present invention to provide an ankle support that exposes particular muscles associated with the foot and ankle.

It is a further object of the present invention to provide an ankle support which does not inhibit blood flow.

It is still another object of the present invention to provide an ankle sport which is relatively inexpensive.

It is still further object of the present invention provide an ankle support that is easy to use and easy to manufacture.

These and other objects and advantages of the present invention will become apparent from a reading of the attached specification and appended claims.

### BRIEF SUMMARY OF THE INVENTION

The present invention is an ankle support that includes a body that is of a generally cylindrical configuration. The body is formed of a polymeric foam material and is shaped so as to conform to the shape of the human ankle and foot. The body includes a first portion that extends over the heel portion of the foot and a second portion that extends over the ankle and lower portion of the calf. The first portion has an oval opening so as to allow the forward portion of the foot to extend outwardly therefrom. The polymeric foam material that is used in the formation of the body is, in particular, a polyethylene open-cell foam. This polymeric foam material can include a neoprene section embedded thereon or therein.

An endoskeletal structure is provided in association with the body so as to add structural strength to the body. This endoskeletal structure is affixed to the body. In particular, this endoskeletal structure is embedded in the polymeric foam material of the body. The endoskeletal structure is of a stirrup configuration and extends from the top of the second portion of the body downwardly along the first portion and back to the top of the second portion. This endoskeletal structure is a screen material. In particular, in the preferred embodiment, the endoskeletal structure is a polyester screen material. The endoskeletal structure bears against the fibularis longus muscles and against the fibularis brevis muscles.

A slot is formed between forward edges of the body. This slot is central of the forward portion of the second portion of the body. This slot extends vertically so as to expose the extensor tendon.

V-shaped cut-outs are formed on opposite sides of the slot at the lower portion of the slot and above the lower end of the body. The V-shaped cut-outs each have a wide end opening to the slot. The V-shaped cut-outs expose the inferior extensor retinaculum.

There is an opening at the rear of the body opposite the slot. This opening at the rear of the body exposes the Achilles tendon. This opening at the rear of the body has rounded corners. A neoprene surface is formed at the bottom of the body on the first portion of the body. In particular, this neoprene surface is located below the heel of a person wearing the ankle support of the present invention.

A wrap extends around the exterior of the body. The wrap is of a removable material. This wrap extends so as to cover the slot at the front of the body. The wrap can be formed of athletic tape, elastic material, or an Ace bandage.

The ankle support of the present invention allows for a tight wrap without cutting off circulation, provides minimal inhibition of motion of the ankle, and prevents the inversion or eversion of the ankle. A stirrup system is embedded in the foam so as to prevent hyperextension or rolling-over of the foot. The openings, slots and grooves correspond to the arteries, veins, and tendons so as to allow the wrap to bridge and not place direct pressure to blood flow or constrict the tendons.

In an alternative embodiment of the present invention, an ankle support is provided that includes a body that is of generally cylindrical configuration. The body is formed of a polymeric foam material and is shaped so as to conform to the shape of a human ankle and foot. The body includes a first portion that extends over the heel portion of the foot and a second portion that extends over the ankle and lower portion of the calf. The first portion has an oval opening so as to allow the forward portion of the foot to extend outwardly therefrom.

In this alternative embodiment, an endoskeletal structure is provided in association with the body so as to add structural strength to the body. This endoskeletal structure is affixed to the body. In particular, this endoskeletal structure is embedded in the polymeric foam material of the body. The endoskeletal structure is of a stirrup configuration and extends from the top of the second portion of the body downwardly substantially over the sides and bottom of the first portion and back to the top of the second portion. This end of skeletal structure is of a screen material.

A slot is formed between forward edges of the body of this alternative embodiment of the present invention. The slot extends vertically so as to expose extensor tendons of the ankle and foot. Small openings or formed on opposite sides of the slot near the lower portion of the slot and above the lower end of the body. These openings each have a wide end opening to the slot. These openings expose the dorsiflexion and plantar flexion tendons. A generally vertical open area extends upwardly from at least one of these openings. This generally vertical open area extends in parallel relationship to the slot. This generally vertical open area is adapted to extend over and anterior tibial vein. The lower portion of the slot that extends through the first portion of the body exposes the dorsal pedis vein. The portion of the slot extending along the second portion of the body exposes the anterior tibial vein.

In this alternative embodiment of the present invention an opening is formed at the rear of the body opposite to the slot. The opening is adapted to expose an Achilles tendon of the ankle. A wrap can be placed around the exterior of the body so as to extend over the slot.

This foregoing Section is intended to describe, with particularity, the preferred embodiments of the present invention. It is understood that modifications to these preferred embodiments can be made within the scope of the present claims. As such, this Section should not to be construed, in any way, as limiting of the broad scope of the present invention. The present invention should only be limited by the following claims and their legal equivalents.

### BRIEF DESCRIPTION OF THE SEVERAL VIEWS OF THE DRAWINGS

FIG. 1 is a perspective view showing the ankle support of the present invention as applied over a human foot and ankle.

FIG. 2 is a perspective view showing the ankle support of the present invention as covered by a tight wrap.

FIG. 3 is a rear view of the ankle support of the present invention.

FIG. 4 is an upper perspective view showing an alternative embodiment of the ankle support of the present invention.

### DETAILED DESCRIPTION OF THE INVENTION

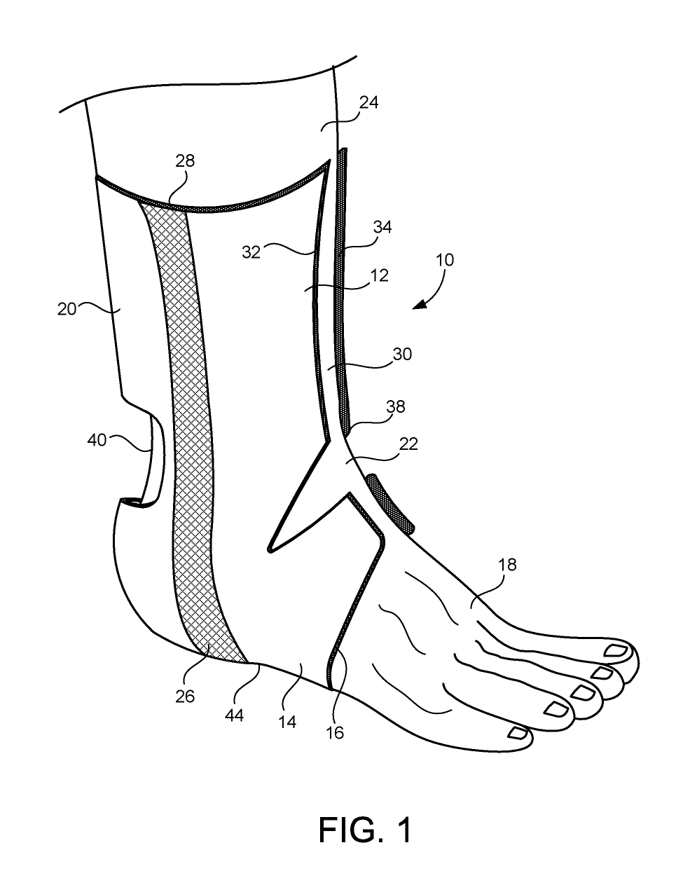

Referring to FIG. 1, there is shown the ankle support 10 in accordance with the preferred embodiment of the present invention. The ankle support 10 includes a body 12 that is configured into a generally cylindrical configuration. The body 12 is formed of a polymeric foam material. In particular, in the preferred embodiment the present invention, this polymeric foam material is a polyethylene open-cell foam. Neoprene can be incorporated into the polymeric foam material for added flexibility and support. The body 12 conforms to the shape of the human ankle and foot. The body 12 includes a first portion 14 that extends over and around the heel portion of the foot. There is an oval opening 16 at the forward edge of the first portion 14 of the body 12 so as to allow the forward portion 18 of the foot extend outwardly therethrough. A second portion 20 of the body 12 extends over the ankle 22 and lower calf 24 of the human leg. The body 12 can have grooves on an interior surface thereof. The grooves that are formed on the interior surface correspond to veins and arteries associated with the ankle.

An endoskeletal structure 26 is affixed to the body 12 in the form of a stirrup-like structure. This endoskeletal structure 26 serves to add strength to the sides of the body 12. In particular, the endoskeletal structure 26 is embedded in the polymeric foam material of the body 12. The endoskeletal structure 26 will extend from the top 28 of the body 12 and downwardly along the sides of the body 12. Ultimately, the endoskeletal structure 26 will extend around the bottom of the first portion 14 of the body 12 and below the bottom of the heel of the foot 18. The skeletal structure 26 will the extend upwardly along the opposite side of the body 12. This endoskeletal structure 26 is in the nature of a screen material. In particular, the screen material can be formed of polyester material. The endoskeletal structure 26 will bear against the fibularis longus muscles and the fibularis brevis muscles. As such, the endoskeletal structure 26 will provide proper support to these muscles.

A slot 30 is located between the forward edges 32 and 34 of the body 12. The slot 30 extends centrally of the forward portion of the second portion 20 of the body 12. The slot 30 will extend generally vertically so as to expose the extensor tendons.

A pair of V-shaped cut-outs 36 and 38 extend outwardly of and open to the slot 30. These V-shaped cut-outs 38 are on opposite sides of the slot 30 and are located generally at the lower portion of the first portion 20 of the body 12 and in a location generally above the foot 18. Each of the V-shaped cut-outs 36 and 38 have a wide end that opens to the slot 30. The V-shaped cut-outs 36 and 38 expose the inferior extensor retinaculum. These V-shaped cutouts 36 and 38 also enhance the flexibility of the ankle support 10 during foot and ankle movement. As such, this will allow the ankle 22 to properly hinge.

There is an opening 40 at the rear of the body 12. This opening exposes the Achilles tendon. It can be seen that the opening 40 has generally rounded edges. Once again, this opening 40 further enhances the flexibility of the ankle support 10 during foot movement while not constricting or applying pressure to the Achilles tendon.

A neoprene surface 44 can be can be formed at the bottom of the body 12 and below the heel of the foot 18. The neoprene surface will enhance the durability of the ankle support 10 in the location where forces are applied by the heel against a shoe or other underlying surface.

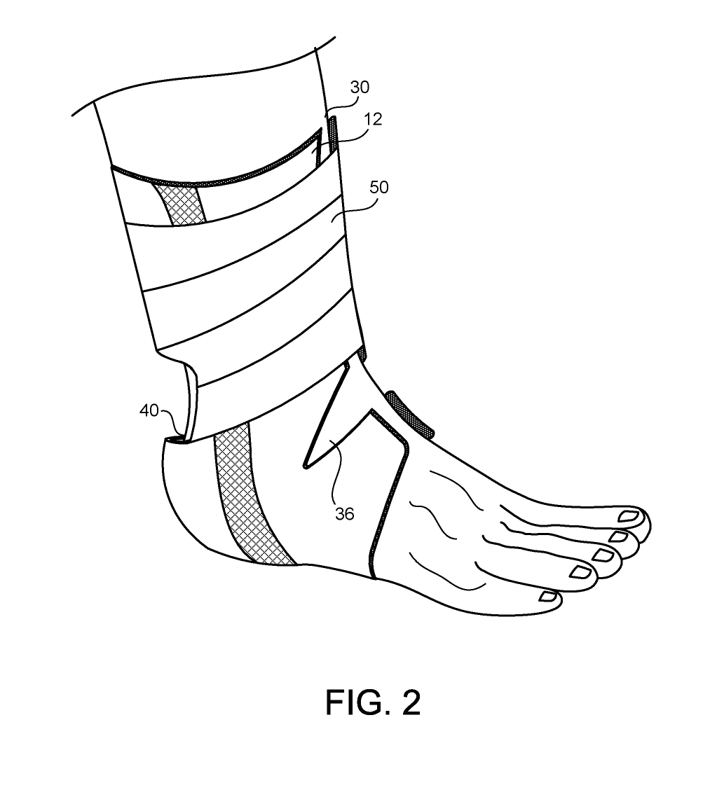

FIG. 2 illustrates that there is a wrap 50 that extends around the exterior of the body 12. After the ankle support 10 is applied onto the ankle 22 in the foot 18, the wrap 50 can be wrapped around the exterior of the body 12. Wrap 50 will extend over the slot 30 and can extend, if desired, over the opening 40. It can further extend over the V-shaped cut-outs 36 and 38, if desired. The wrap 50 can be in the nature of an elastic material, such as an athletic tape or an Ace bandage. The wrap 50 will serve to secure the ankle support 10 securely around the ankle and foot of the wearer. The wrap 50 can be utilized so as to apply a desired amount of pressure to the ankle and foot. Since the wrap 50 extends over the top of the slot 30 and over the top of the opening 40, it will not impair blood flow or adversely affect the muscles or tendons in the area of the ankle.

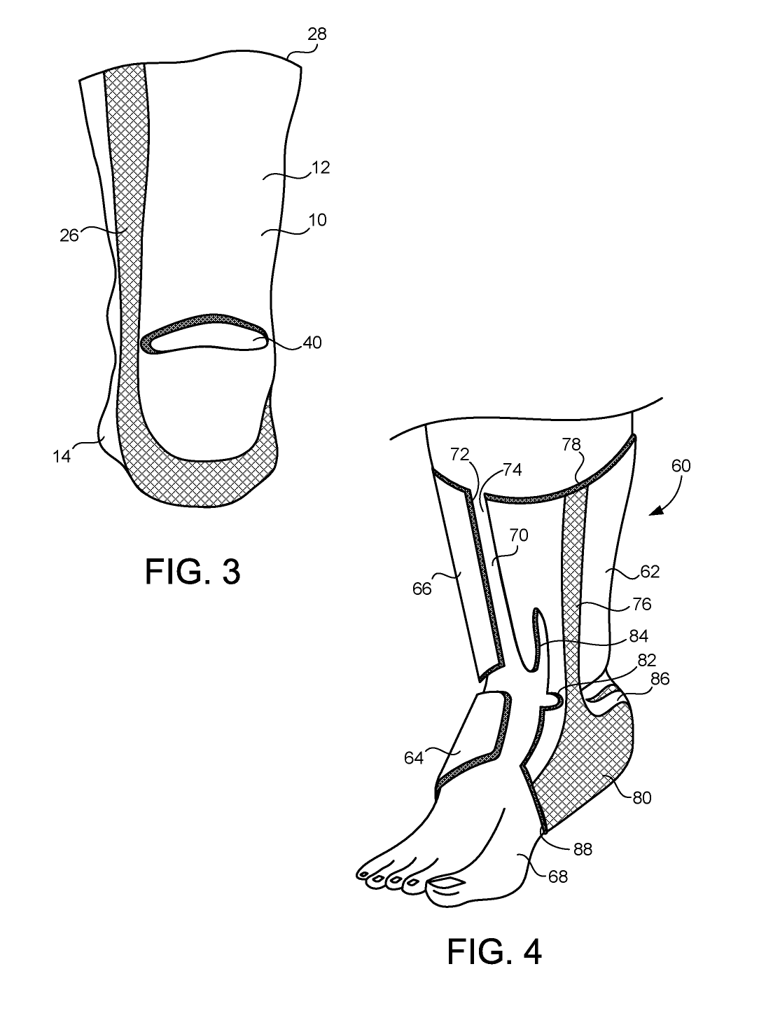

Referring to FIG. 3 a rear view of the ankle support 10 of the present invention is shown. In FIG. 3, it can be seen that the endoskeletal structure 26 extends across and over the bottom of the first portion 14 and along sides of this first portion 14. Ultimately, the endoskeletal structure 26 will extend up to the top 28 of the body 12. Opening 40 is formed at the rear of the first portion 14. Opening 40 exposes the Achilles tendon. This opening 40 can have a generally triangular configuration with rounded edges. This opening 40 enhances the flexibility of the ankle support 10 during foot movement while not constricting or applying pressure to the Achilles tendon.

FIG. 4 shows an alternative embodiment of the ankle support 60 in accordance with the teachings of the present invention. The ankle support 60 includes a body 62 having a generally cylindrical configuration and adapted to conform to a shape of the human ankle. The body 62 has a first portion 64 and a second portion 66. The first portion 64 is integrally formed with the second portion 66. The body is formed of a flexible material, such as a polymeric foam material. The first portion 64 is adapted to extend over a heel portion of the human foot 68. The second portion 66 is adapted to extend over the human ankle and over a lower portion of a human calf. The body has a slot 70 formed between edges 72 and 74 at a forward portion of the body 62.

An endoskeletal structure 76 is affixed to the body 62 and extends along sides of said body 62 and along said second portion 66 of said body 62. The endoskeletal structure 76 also extends along sides of the first portion 64 and along a bottom of the first portion. In FIG. 4 (with reference to FIG. 3), the skeletal structure 76 is a continuous piece of material and extends to the top edge 78 of the body 62. The lower portion 80 of the endoskeletal structure 76 extends substantially over the sides and bottom of the first portion 64. As such, this lower area 80 of the endoskeletal structure 76 will substantially cover the heel portion of the foot 68. As with the previous embodiment, the endoskeletal structure 76 is a continuous strip of screen material and bears against a fibula longus muscle and against a fibularis brevis muscle.

The body 62 has a pair of openings 82 formed on opposite sides of the slot 70 adjacent the lower area of the second portion 66 of the body 62. A wide end of the openings 82 opens to the slot 70. The openings 82 expose the dorsiflexion and plantar flexion tendons. A generally vertical open area 84 extends upwardly from one of the openings 82 or from the slot 70. This generally vertical open area 84 extends in parallel relationship to the upper portion of the slot 70. This generally vertical open area 84 is adapted to extend over an posterior tibial vein. An opening 86 is formed at the rear of the body 62 opposite to the slot 70. This opening 86 is adapted to expose an Achilles tendon of the ankle. The first portion 64 has an opening 88 that allows a forward portion of the human foot 68 to extend therethrough. A wrap can be applied around the exterior of the body 62 so as to extend over the slot 70.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. An ankle support article comprising:
a body having a generally cylindrical configuration and adapted to conform to a shape of a human ankle, said body having a first portion and a second portion integrally formed together of a flexible material, said first portion adapted to extend over a heel portion of a human foot, said second portion adapted to extend over the human ankle and over a lower portion of a human calf, said body having a slot formed between edges at a forward portion of said body.

2. The ankle support article of claim 1, further comprising:
an endoskeletal structure affixed to said body and extending along sides of said body along said second portion of said body.

3. The ankle support article of claim 2, said endoskeletal structure extending along sides of said first portion and along a bottom of said first portion.

4. The ankle support article of claim 3, said endoskeletal structure being continuous and extending to a top of said body, said endoskeletal structure extending substantially over the side and the bottom of said first portion.

5. The ankle support article of claim 1, said endoskeletal structure being a continuous strip of screen material and adapted to bear thereagainst a fibularis longus muscle and against a fibularis brevis muscle.

6. The ankle support article of claim 1, said body being formed of a polymeric foam material.

7. The ankle support article of claim 1, further comprising:
a pair of generally V-shaped cut-outs formed on opposite sides of said slot adjacent a lower area of said second portion of said body.

8. The ankle support article of claim 7, each of said pair of V-shaped cut-outs having a wide end opening to said slot.

9. The ankle support of claim 8, further comprising:
a generally vertical open area that extends upwardly from at least one of said pair of V-shaped cut-outs, said generally vertical open area extending in parallel relation to said slots, said generally vertical open area adapted to extend over an anterior tibial vein.

10. The ankle support article of claim 1, said slot extending vertically and adapted extensor tendons.

11. The ankle support article of claim 1, further comprising:
an opening formed at the rear of said body opposite to said slot, said opening adapted to expose an Achilles tendon of the ankle.

12. The ankle support article of claim 1, further comprising:
a wrap wrapped around an exterior of said body so as to extend over said slot.

13. The ankle support article of claim 1, said first portion having an oval opening adapted to allow a forward portion of the human foot to extend therethrough.

14. An ankle support assembly comprising:
a body having a generally cylindrical configuration and adapted to conform to a shape of a human ankle, said body having a first portion and a second portion integrally formed together, said first portion adapted to extend over a heel portion of a human foot, said second portion adapted to extend over the human ankle and over a lower portion of a human calf, said body having a slot formed between edges at a forward portion of said body; and
a wrap wrapped around in exterior said body so as to extend over said slot.

15. The ankle support assembly of claim 14, further comprising:
an endoskeletal structure affixed to said body and extending along sides of a second portion of said body, said endoskeletal structure extending along sides and a bottom of said first portion.

16. The ankle support assembly of claim 14, further comprising:
a pair of V-shaped cut-outs formed on opposite sides of said slot adjacent a lower area of said second portion of said body, each of said pair of V-shaped cut-outs having a wide end opening to said slot.

17. An ankle support article comprising:
a body having a generally cylindrical configuration and adapted to conform to a shape of the human ankle, said body having a first portion and a second portion integrally formed together of a flexible material, said first portion adapted to extend over a heel portion of the human foot, said second portion adapted to extend over the human ankle and over a lower portion of a human calf; and
an endoskeletal structure affixed to said body and extending along sides of said second portion of said body, said endoskeletal structure extending along sides of said first portion and along a bottom of said second portion, said endoskeletal structure being continuous.

18. The ankle support article of claim 17, said body having a slot formed between edges at a forward portion of said body.

19. The ankle support article of claim 18, further comprising:
a wrap wrapped around in exterior of said body so as to extend over said slot.
